(19) United States
(12) Patent Application Publication
(10) Pub. No.: US 2010/0190237 A1
(43) Pub. Date: Jul. 29, 2010

(54) COMPOSITIONS AND METHODS FOR STORAGE OF BACTERIAL SUSPENSIONS

(75) Inventor: Nirit Ulitzur, Kiryat-Tivon (IL)

Correspondence Address: Fleit Gibbons Gutman Bongini & Bianco PL, 21355 EAST DIXIE HIGHWAY, SUITE 115, MIAMI, FL, 33180, US

(73) Assignee: CHECK LIGHT LTD., Qiryat-Tiv'on (IL)

(21) Appl. No.: 12/676,944

(22) PCT Filed: Sep. 18, 2008
(86) PCT No.: PCT/IL08/01254

§ 371 Date: Mar. 8, 2010

Related U.S. Application Data
(60) Provisional application No. 60/960,312, filed on Sep. 25, 2007.

(51) Int. Cl. C12N 1/20

(52) U.S. Cl. 435/252.1; 435/253.6

(57) ABSTRACT

A medium composition is provided. The medium composition comprising about: NaCl/KCl 1.0-3.0%; Inositol/lactose/trehalose/dextran 0.5-7.0%; Mg/Ca 0.0-300 mM; Yeast Extract/Casamino acids 0.01-0.05%; BSA/egg albumin 0.02-1.0%; and Ethanol/methanol/propanol 0.1-3.5%.

### FIELD AND BACKGROUND OF THE INVENTION

The present invention relates to medium compositions and methods of using same such as for storage of bacteria in ways that preserve desired characteristics of the stored bacteria.

Analysis of emitted light from luminescent bacteria is an effective and rapid method for hygiene monitoring and toxicity testing of water or other fluids. In assays based upon luminescent bacteria, a reduction of the light emitted by the bacteria is proportional to the presence of toxic materials in the sample. Assays of this type can be sensitive, quantitative and rapid.

Previously available systems for assays of toxicity based upon analysis of emitted light from luminescent bacteria typically rely on providing either an aliquot of an active bacterial culture or an aliquot of reconstituted bacteria (e.g. rehydration of a lyophilized culture). Aliquots of active bacterial cultures can be provided from either a closed culture or an open growth system.

Bacteria, including luminescent bacteria, grown in closed culture exhibit various stages of growth: lag phase, acceleration phase, exponential phase, retardation phase and death phase. Closed batch culture vessels contain a mixture of microorganisms at various stages of the cell cycle. The number of microorganisms will increase at varying rates during the different phases of growth of a culture and the culture will eventually decline and die. As the number of organisms in the culture increases the environment in which they live changes. Many cellular components, such as ATP, DNA and proteins alter in response to these changes and thus the differences between individuals in their biochemical responses (e.g. luminescence) varies.

In an open growth system there is a continuous input of growth substrates (medium) and removal of waste products, cells and unused substrate. Parameters such as pH, oxygen, temperature etc. can be monitored during growth with any changes automatically compensated for, for example by the addition of substances to stimulate or retard growth. In open growth or continuous flow cultures, the exponential growth phase is prolonged indefinitely as additions to, and removal from, the culture take place continuously.

Current practice in quasi-continuous toxicity testing typically employs rehydrated lyophilized suspensions of bacteria. U.S. Pat. No. 5,801,052 describes an apparatus for reconstituting dried bacteria.

Cultivation of luminous bacteria is described, for example, in Zavoruev and Mezhevikin (1983) Prikl. Biokhim. Mikrobiol. 19, pp. 564-568A recombinant bacterial biosensor systems has been proposed [D′ Souza, 2001 Biosens Bioelectron. 2001 August; 16(6):337-53]

U.S. Pat. No. 6,949,382 describes a cell culture media including inositol and magnesium.

Rupani et al. [Biotechnol Prog (1886) 12:367-392] describe use of ethanol as an inducer of bacterial growth.

### SUMMARY OF THE INVENTION

In an exemplary embodiment of the invention, there is provided a medium composition, the composition comprising about:

| | |
|---|---|
| NaCl/KCl (%) | 1.0-3.0; |
| Inositol/lactose/trehalose/dextran (%) | 0.5-7.0; |
| Mg/Ca (mM) | 0.0-300; |
| Yeast Extract/Casamino acids (%) | 0.01-0.05; |
| BSA/egg albumin (%) | 0.02-1.0; and |
| Ethanol/methanol/propanol (%) | 0.1-3.5. |

Optionally, the medium composition comprises about:

| | |
|---|---|
| NaCl/KCl (%) | 2.0-3.0; |
| Inositol/lactose/trehalose/dextran (%) | 5.0-7.0; |
| Mg/Ca (mM) | 1.0-200; |
| Yeast Extract/Casamino acids (%) | 0.01-0.05; |
| BSA/egg albumin (%) | 0.02-0.1; and |
| Ethanol/methanol/propanol (%) | 1.0-3.5. |
| NaCl/KCl (%) | 2.0; |
| Inositol/lactose/trehalose/dextran (%) | 5.0; |
| Mg/Ca (mM) | 2.0; |
| Yeast Extract/Casamino acids (%) | 0.05; |
| BSA/egg albumin (%) | 0.05; and |
| Ethanol/methanol/propanol (%) | 1.5. |

Optionally, the medium composition comprises an antibiotic.

Optionally, the medium composition is provided as a liquid.

In an exemplary embodiment of the invention, there is provided a bacterial suspension comprising a medium composition as described above and bacteria suspended therein.

Optionally, the bacteria comprise luminescent bacteria.

In an exemplary embodiment of the invention, there is provided a method of preparing a bacterial suspension as described above comprising:

wetting fibers with a bacterial culture to form wetted fibers;

lyophilizing the wetted fibers to form a plug; and

immersing the plug in the medium composition to form the bacterial suspension.

In an exemplary embodiment of the invention, there is provided a method of storing bacteria, the method comprising:
(a) suspending bacteria in the medium composition of any of claims 1-3 so as to form a bacterial suspension; and
(b) storing said bacterial suspension under conditions for maintaining bacteria viability and preventing bacterial expansion.

Optionally, suspending comprises immersing lyophilized bacteria on fibers in the medium composition.

Optionally, the bacteria comprise luminescent bacteria.

Optionally, the maintaining bacteria viability and preventing bacterial expansion is for at least 2 weeks at 2 to 8 degrees centigrade.

Optionally, the medium composition comprises at least one antibiotic. Optionally, the at least one antibiotic comprises at least one compound selected from the group consisting of chloramphenicol and ampicillin.

Optionally, the at least one antibiotic is provided at a concentration of about 15 mg/L.

Optionally, the conditions further comprise maintaining luminescence of said luminescent bacteria.

In an exemplary embodiment of the invention, there is provided an article of manufacture, comprising:
(a) a medium composition according to any of claims 1-3; and
(b) instructions for suspending bacteria in the medium composition to form a bacterial suspension and storing the bacteria at −2 to +8 degrees centigrade.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified flow diagram of a method for storing a bacterial suspension at refrigeration temperatures for a prolonged period of time according to an exemplary embodiment of the invention;

FIG. 2 is a graph of luminescence in relative light units as a function of time illustrating the effect of different concentrations of albumin;

FIG. 3 is a graph of luminescence in relative light units as a function of time illustrating the effect of different concentrations of alcohol;

FIG. 4 is a graph of luminescence in relative light units as a function of time illustrating the effect of different concentrations of sugar;

FIG. 5 is a graph of luminescence in relative light units as a function of time illustrating the effect of different concentrations of divalent cations;

FIG. 6 is a graph of luminescence in relative light units as a function of time illustrating the effect of different concentrations of monovalent salt;

FIG. 7 is a graph of luminescence in relative light units as a function of time illustrating the effect of different pH;

FIG. 8 is a graph of luminescence in relative light units as a function of time illustrating the effect of different concentrations of a protein mixture; and

FIG. 9 is a graph of luminescence in relative light units as a function of time illustrating the effect of different antibiotics.

### DESCRIPTION OF THE PREFERRED EMBODIMENTS

Embodiments of the invention relate to medium compositions which can in some embodiments be implemented in storing a bacterial suspension for a period of at least two weeks, optionally three, four, or five weeks, at refrigeration temperatures while maintaining a selected property of the suspension within a desired range. In an exemplary embodiment of the invention, the method employs a medium with a novel set of components at selected concentrations.

Specifically, some embodiments of the invention can be used to maintain luminescence of bacteria in a dormant suspension. In an exemplary embodiment of the invention, the luminescence is useful in performance of assays for one or more toxic substances in liquid (e.g. water) samples.

The principles and operation of a method and/or apparatus according to exemplary embodiments of the invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The present inventor has synthesized, through laborious experimentation and screening a novel medium composition which can be used to preserve luminescence of bacteria in a desired range for a prolonged period of time.

The medium solution comprises a monovalent salt, a sugar, optionally a source of divalent cations, a protein mixture, an albumin and an alcohol. Optionally, the medium comprises at least one antibiotic. Optionally, the protein mixture comprises non-protein ingredients such as vitamins and/or fatty acids and/or lipids.

In an exemplary embodiment of the invention, the monovalent salt includes NaCl and/or KCl. Optionally, the monovalent salt is provided at a concentration of about 1 to 3%, optionally about 2%. In an exemplary embodiment of the invention, 2% NaCl is employed.

In some exemplary embodiments of the invention, a monovalent salt concentration of about 0.8% is useful. Optionally, 0.8% NaCl is useful in preserving luminescence of genetically modified E. coli.

In an exemplary embodiment of the invention, the sugar is selected from the group consisting of inositol, lactose, trehalose and dextran. Optionally, the sugar is provided at a concentration of at least about 0.5% but not more than about 7%. In an exemplary embodiment of the invention, 5 to 7% inositol is employed.

In an exemplary embodiment of the invention, the divalent cations comprise magnesium ions. Optionally, the magnesium ions are provided at a concentration of about 1 mM to about 200 mM.

In an exemplary embodiment of the invention, the protein mixture comprises yeast extract and/or casamino acids. Optionally, the protein mixture comprises yeast extract at a concentration of at least about 0.01% but not exceeding about 0.05%.

In an exemplary embodiment of the invention, the albumin comprises bovine serum albumin (BSA). Optionally, the BSA is provided at a concentration of at least about 0.02% but not exceeding about 1.0%.

In an exemplary embodiment of the invention, the alcohol comprises an alcohol selected from the group consisting of ethanol, methanol and propanol. Optionally, the alcohol comprises ethanol is provided at a concentration of at least about 0.1% but not exceeding about 5%, optionally about 1.5%.

In those embodiments of the invention that employ antibiotics, chloramphenicol and/or ampicillin can be useful, optionally at a concentration of about 15 mg/L.

Thus, according to one exemplary embodiment of the invention there is provided a medium composition comprising a monovalent salt (e.g. NaCl and/or KCl at 1.0-3.0%, a sugar (e.g. inositol/lactose/trehalose/dextran at 0.5-7.0%, a divalent cation (e.g. Mg or Ca) at 0-300 mM, a source of amino acids (e.g. yeast extract or casamino acids) at 0.01-0.05%, an albumin (e.g. BSA or egg albumin) at 0.02-1.0% and an alcohol (e.g. ethanol, methanol or propanol) at 0.1-3.5%.

In another exemplary embodiment of the invention, the medium composition comprises one a monovalent salt (e.g. NaCl and/or KCl at 2.0-3.0%, a sugar (e.g. Inositol/lactose/trehalose/dextran at 5.0-7.0%, a divalent cation (e.g. Mg or Ca) at 1.0-200 mM, a source of amino acids (e.g. yeast extract or casamino acids) at 0.01-0.05%, an albumin (e.g. BSA or egg albumin) at 0.02-1.0% and an alcohol (e.g. ethanol, methanol or propanol) at 1.0-3.5%.

In another exemplary embodiment of the invention, the medium composition comprises a monovalent salt (e.g. NaCl and/or KCl at about 2%, a sugar (e.g. Inositolinositol/lactose/trehalose/dextran at about 5%, a divalent cation (e.g. Mg or Ca) at about 2 mM, a source of amino acids (e.g. yeast extract or casamino acids) at about 0.05%, an albumin (e.g. BSA or egg albumin) at about 0.05% and an alcohol (e.g. ethanol, methanol or propanol) at abut 1.5%.

According to various embodiments of the invention, the medium composition can be provided as a solid or as a liquid. For solid compositions agar, agarose or other solidifying agents can be used.

The medium compositions described herein may be produced from sterile reagents. Alternatively or additionally, medium compositions can be sterilized following production. Once the composition are at hand, bacteria can be suspended therein.

Listed components of the medium composition are available commercially from a variety of sources. For example, NaCl, KCl, MgCl₂, CaCl₂, inositol, lactose, trehalose, dextran, methanol, ethanol, propanol, yeast extract, BSA, egg albumin, are available from, for example, Sigma-Aldrich Co. (St. Louis Mo., USA). Casamino acids are available, for example, from Becton Dickinson and Co. (Franklin Lakes N.J., USA).

A shown in examples 1-13 the medium compositions illustrated herein can be used for toxicant assays based upon bacterial luminescence.

Thus, according to another aspect of the invention there is provided a method of storing bacteria. Medium disclosed hereinabove and described in examples hereinbelow can be useful for storing naturally luminescent bacteria (e.g. Photobacterium leiognathi, Vibrio fischeri, Vibrio harveyi) and/or bacteria expressing exogenous genes (e.g. E. coli harboring the luminescence system of V. fischeri). Optionally, toxin sensitive promoters are used to regulate luminescence genes.

In some exemplary embodiments of the invention biochemical properties not related to luminescence are employed (e.g. color and/or an enzymatic activity) as a basis for an assay.

In an exemplary embodiment of this aspect of the invention, a lyophilized pellet of bacteria is suspended. Optionally, a wool or cotton fiber may be soaked in the bacterial culture, lyophilized and maintained for long term storage until suspended in the storage solution. In an exemplary embodiment of the invention, dispersion of the bacteria among fibers contributes to ease of resuspension. Alternatively, a bacterial culture can be precipitated (e.g. by centrifugation) and suspended in the above-described medium solution to form the bacterial suspension.

The bacterial suspension can then be stored at −2 to +8, optionally −1 to +6, optionally +2 to +5, optionally about 4 degrees centigrade for a period of at least two weeks while maintaining a selected property of the suspension in a desired range. In an exemplary embodiment of the invention, the selected property includes a degree of luminescence of the bacterial suspension in case luminescent bacteria are used. Alternatively, or additionally, other biochemical properties (e.g. color or a selected enzymatic activity) can be employed. Optionally, the medium maintains the selected property of the suspension in a desired range for three, four or five weeks or intermediate or longer periods of time.

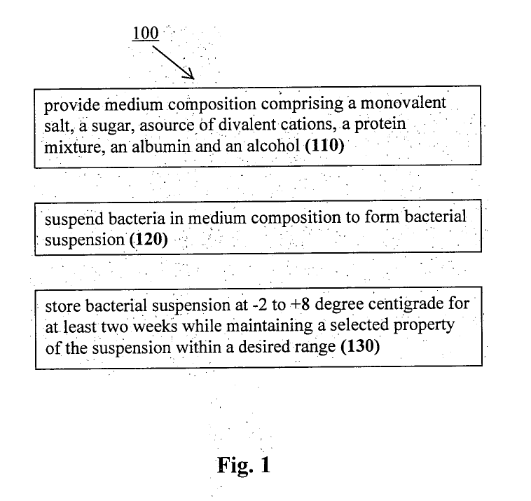

Referring now to the drawings, FIG. 1 illustrates a method 100 of storing bacteria. Method 100 includes providing 110 a medium composition comprising a monovalent salt, a sugar, a source of divalent cations, a protein mixture, an albumin and an alcohol. Optionally, the medium composition comprises at least one antibiotic. Optionally, the protein mixture comprises non-protein ingredients such as vitamins and/or fatty acids and/or lipids.

According to the depicted method, bacteria are suspended 120 in the medium composition to form a bacterial suspension. In an exemplary embodiment of the invention, a lyophilized pellet of bacteria is suspended. Optionally, a wool or cotton fiber may be soaked in the bacterial culture, lyophilized and maintained for long term storage until suspended in the medium composition. In an exemplary embodiment of the invention, dispersion of the bacteria among fibers contributes to ease of resuspension. Alternatively, a bacterial culture can be precipitated (e.g. by centrifugation) and suspended in the medium composition to form the bacterial suspension.

The bacterial suspension can then be stored 130 at −2 to +8, optionally −1 to +6, optionally +2 to +5, optionally about 4 degrees centigrade for a period of at least two weeks while maintaining a selected property of the suspension in a desired range. In an exemplary embodiment of the invention, the selected property includes a degree of luminescence of the bacterial suspension. Optionally, the medium composition maintains the selected property of the suspension in a desired range for three, four or five weeks or intermediate or longer periods of time.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. In case of conflict, the patent specification, including definitions, will control. In addition, the materials, methods, and examples are illustrative only and not intended to be limiting.

The phrase “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed composition, device or method.

The term “method” refers to manners, means, techniques and procedures for accomplishing a given task including, but not limited to, those manners, means, techniques and procedures either known to, or readily developed from known manners, means, techniques and procedures by practitioners of microbiology and/or toxicology.

Percentages (%) of chemicals typically supplied as powders or crystals (e.g. salts, sugars and proteins) are W/V (weight per volume) unless otherwise indicated. Percentages (%) of chemicals typically supplied as liquids (e.g. alcohols) are V/V (volume per volume) unless otherwise indicated.

As used herein the phrase “selected property of the suspension in a desired range” refers to viability such as can be manifested by reconstituted proliferation upon incubation in suitable growth conditions, co-culturing, gene expression, growth/expression induction (e.g., IPTG induced) as well as reporter activity (e.g., luminescence), desired catalytic activity, such as probiotic activity or fossil fuel hydrolysis.

The terms “include” and “have”, and their conjugates, as used herein mean “including but not necessarily limited to”

In some exemplary embodiments of the invention, the medium composition is provided as part of an article of manufacture. Optionally, the medium is provided in sterile and/or measured units. Optionally, each measured unit is provided in an individual container.

Optionally, the medium is provided with instructions for suspending bacteria therein to form a bacterial suspension which maintains a selected property of the suspension within a desired range at −2 to +8 degree centigrade for a period of at least two to five weeks. In an exemplary embodiment of the invention, the selected property comprises a degree of luminescence.

In other exemplary embodiments of the invention, the medium is provided with bacteria suspended therein and is supplied refrigerated with labeling indicating that the solution maintains a selected property of the suspension within a desired range at −2 to +8 degree centigrade for a period of at least two to five weeks. In an exemplary embodiment of the invention, the selected property comprises a degree of luminescence.

In an exemplary embodiment of the invention, each measured unit is provided in a container configured for attachment to an automated toxicity assay apparatus. Optionally, measured units are prepared to accommodate a specific number of assays. Typically, a toxicity assay uses 10 to 50 μl of bacterial suspension (e.g. in a 1 ml to 5 ml reaction volume) so that 1 ml is sufficient for 20 to 100 assays. Assuming that each assay lasts 15 to 60 minutes, a 50 ml, measured unit is sufficient for 10 to 50 days of testing. In an exemplary embodiment of the invention, measured units of 50, 100, 150, 200, 250, 300 or 400 ml (or intermediate volumes) are provided and provide aliquots of bacterial suspension to multiple testing chambers in a single apparatus.

Optionally, multiple measured units are provided in a single apparatus and used sequentially. In an exemplary embodiment of the invention, two or more measured units used sequentially contribute to an extension in a period of time between maintenance visits to an automated apparatus.

It is expected that during the life of this patent many relevant influences of toxicants on bacteria will be developed and the scope of the term assay is intended to include all such new technologies a priori.

As used herein the term “about” refers to ±5% or ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### Example 1

### General Titration Protocol

In order to identify storage conditions suitable for a long term medium for a suspension of luminescent bacteria (e.g. Photobacterium leiognathi, Vibrio fischeri, Vibrio harveyi) different concentrations of monovalent salts, sugars, sources of divalent cations, protein mixtures, albumins and alcohols were tested.

NaCl was selected as a representative monovalent salt because it is commonly employed in bacterial culture media. It is believed that other monovalent salts, including, but not limited to, KCl could be employed at similar concentrations.

Magnesium was selected as a representative divalent cation because it is commonly employed in bacterial culture media. In experiments described herein, magnesium was added as MgCl₂. It is believed that other divalent cations, including, but limited to calcium could be employed at similar concentrations.

Because commercial preparation of large amounts of medium is envisioned, commercially available components for each ingredient category were selected.

Ethanol was selected as a representative alcohol although it believed that other alcohols (e.g. methanol or propanol) could be employed.

Yeast extract was selected as a representative protein mixture although it believed that other mixtures (e.g. casamino acids) could be employed.

Inositol was selected as a representative sugar although it believed that other sugars (e.g. lactose, trehalose and dextran) could be employed. Optionally, a sugar is selected in view of a specific type of bacteria that will be suspended and stored in the solution.

Bovine serum albumin (BSA) was selected as a representative albumin although it is believed that other albumins (e.g. egg albumin) could be employed.

Table 1 below summarizes medium components and assayed ranges.

TABLE 1
summary of titrated ranges

| | | | Amount used |
|---|---|---|---|
| | Exemplary | | for titration |
| | component | | of other |
| component type | employed | Titrated range | ingredients |
| monovalent salt | NaCl | NaCl 0.1-4.5% | 2% |
| sugar | Inositol | 0-11%; | 5% |
| source of divalent | Magnesium | 0-1M | 2 mM |
| cations | Chloride | | |
| protein mixture | Yeast extract | 0-0.2% | 0.05% |
| albumin | BSA | 0-3% | 0.05% |
| alcohol | ethanol | 0-5% | 1.5% |

The pH of the medium was also titrated in the range of pH 5.5-8.

Each of the medium components (including pH) was tested separately while all other components were kept at a constant concentration as indicated in Table 1. For titration of other components pH was held constant at 7.2. The effect on bacterial luminescence of each component at each tested concentration (or pH value) was determined over a period of several weeks.

In order to perform the titration of the medium components, a bacterial culture was spread on LA plates at about 300 colonies per plate after over night growth. Colonies were collected from plates with ASW and centrifuged (10000 g, 10 minutes, 4° C.). Harvested bacteria were resuspended in the different solutions to form experimental bacterial suspensions.

Experimental bacterial suspensions were stored at −2° C.-+4° C. Each week an aliquot of each experimental bacterial suspension was drawn and diluted in artificial seawater (ASW: 0.5M NaCl, 50 mM MgSO₄7H₂O, 10 mM CaCl₂2H₂O, 10 mM KCl)) supplemented with 50 ppm yeast extract/glucose and incubated at 26° C.-28° C. Light level was recorded after 30 minutes.

Results for titration of each component are presented hereinbelow. Similar results were obtained using lyophilized bacterial pellets to prepare experimental suspensions (data not shown).

### Example 2

### BSA Titration Results

In order to determine the effect of Albumin on luminescence during long term storage at refrigeration temperatures, BSA was added to medium at concentrations of 0.02, 0.1, 0.4, 0.5, 1 and 3%. Medium with no BSA served as a negative control. Assay of the effect of BSA was as described in Example 1.

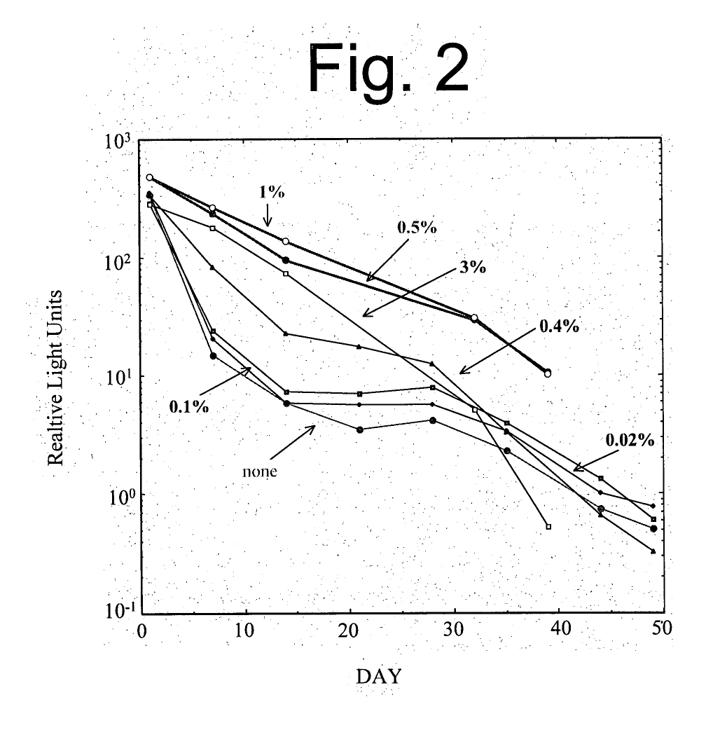

FIG. 2 provides a graphic summary of the results indicating luminescence (in relative light units) as a function of time (in days). The results indicate that 0.5 to 1% BSA slowed the reduction in luminescence better than other tested concentrations. When all other components were optimized 0.05% BSA was selected.

### Example 3

### Ethanol Titration Results

In order to determine the effect of an alcohol on luminescence during long term storage at refrigeration temperatures, ethanol was added to medium at concentrations of 0.1, 1, 1.5, 3, 3.5 and 5%. Medium with no ethanol served as a negative control. Assay of the effect of ethanol was as described in Example 1.

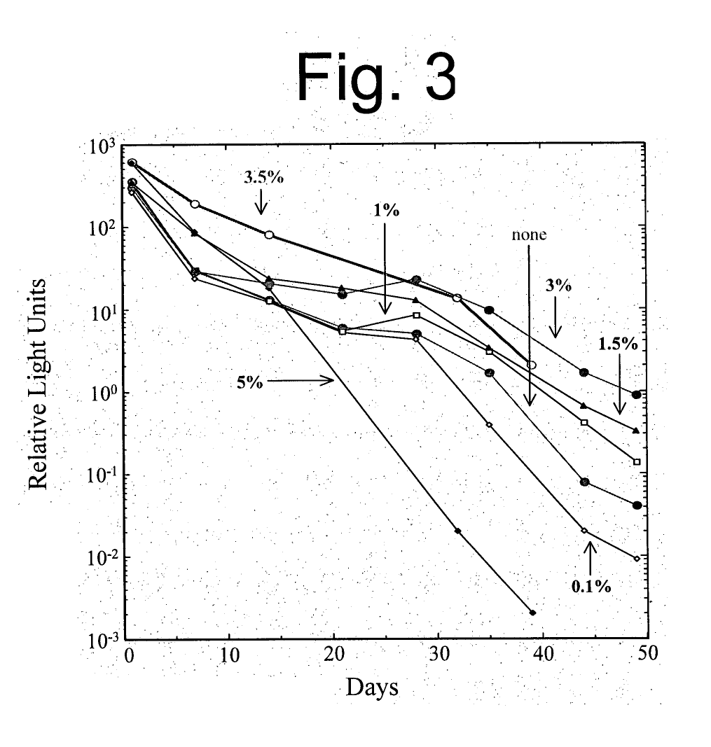

FIG. 3 provides a graphic summary of the results indicating luminescence (in relative light units) as a function of time (in days). The results indicate that 1.5% ethanol or more slowed the reduction in luminescence better than lower tested concentrations.

### Example 4

### Inositol Titration Results

In order to determine the effect of a sugar on luminescence during long term storage at refrigeration temperatures, inositol was added to medium at concentrations of 0.5, 5, 7 and 11%. Medium with no inositol served as a negative control. Assay of the effect of inositol was as described in Example 1.

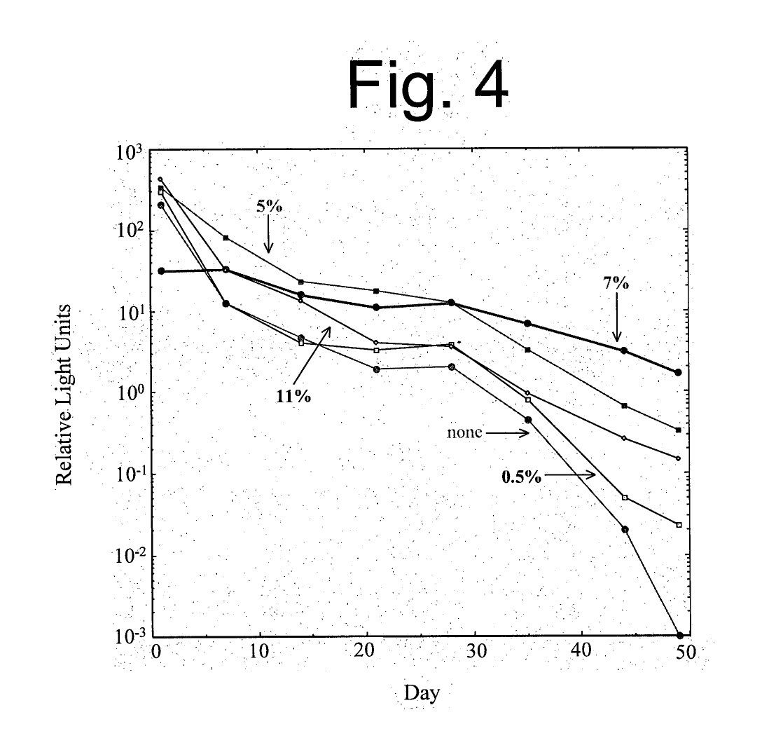

FIG. 4 provides a graphic summary of the results indicating luminescence (in relative light units) as a function of time (in days). The results indicate that 5% inositol slowed the reduction in luminescence better than other tested concentrations. 7% inositol had a similar effect at 28 to 50 days but was less effective than 5% inositol at shorter times.

### Example 5

### Magnesium Titration Results

In order to determine the effect of divalent cations on luminescence during long term storage at refrigeration temperatures, magnesium (MgCl₂) was added to medium at concentrations of 1, 50, 200, 300, 600 and 1000 mM. Medium with no magnesium served as a negative control. Assay of the effect of magnesium was as described in Example 1.

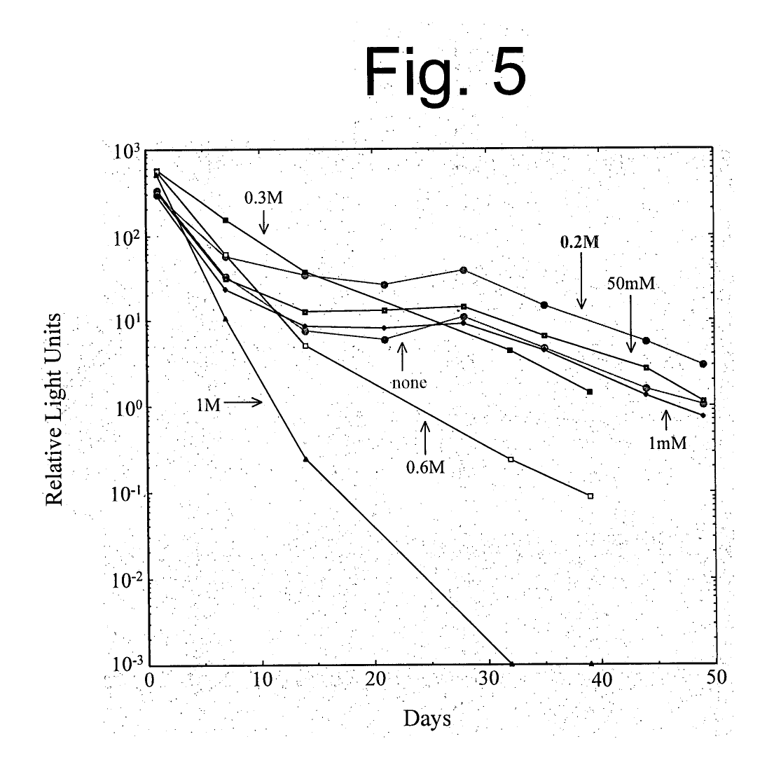

FIG. 5 provides a graphic summary of the results indicating luminescence (in relative light units) as a function of time (in days). The results indicate that 200 mM magnesium slowed the reduction in luminescence better than other tested concentrations. 300 mM magnesium was most effective at 0 to 14 days, but produced lower luminescence than the control solution by day 28. When all other components were optimized 2 mM magnesium was selected.

### Example 6

### NaCl Titration Results

In order to determine the effect of monovalent salts on luminescence during long term storage at refrigeration temperatures, NaCl was added to medium at concentrations of 0.1, 0.5, 2.0, 3.0 and 4.5%. A negative control was not included because very low concentrations of NaCl kill bacteria. Assay of the effect of NaCl was as described in Example 1.

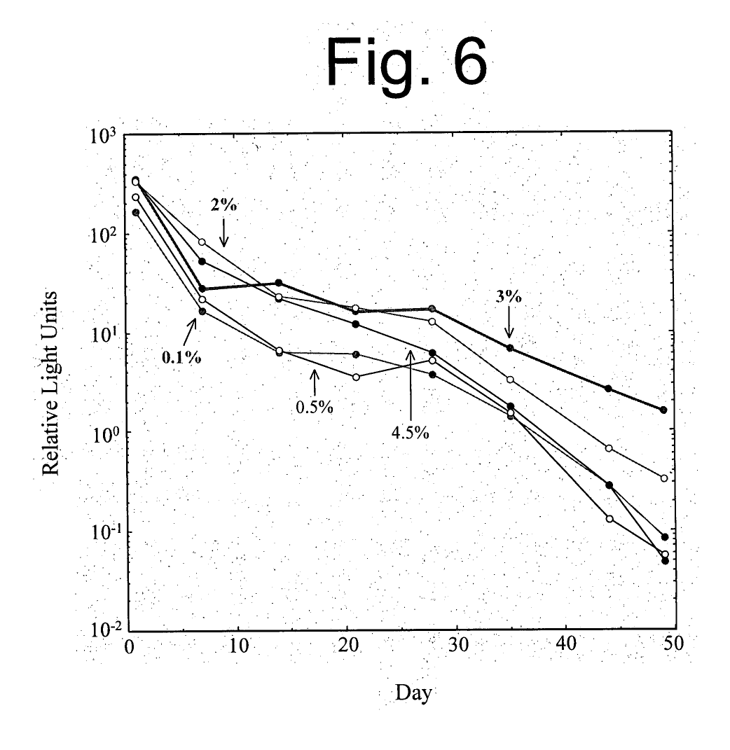

FIG. 6 provides a graphic summary of the results indicating luminescence (in relative light units) as a function of time (in days). The results indicate that 2 to 3% NaCl slowed the reduction in luminescence better than other tested concentrations. 2% NaCl was most effective at 0 to 14 days, but 3% NaCl was more effective after day 28. The two concentrations gave similar results between 14 and 28 days.

### Example 7

### pH Titration Results

In order to determine the effect of pH on luminescence during long term storage at refrigeration temperatures, storage solutions with pH of 5.5, 6.0. 6.5, 7.2, 7.5 and 8.0 were tested. Assay of the effect of pH was as described in Example 1.

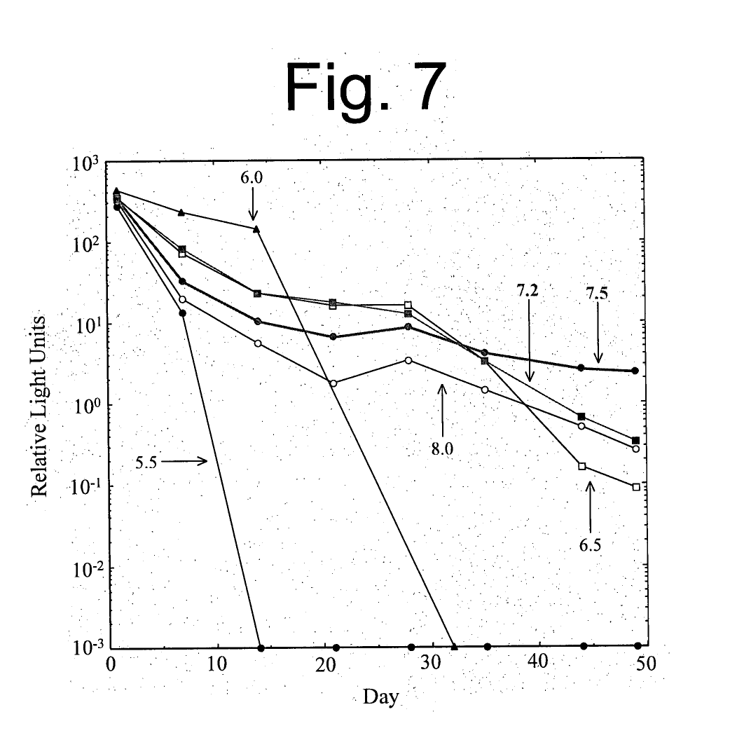

FIG. 7 provides a graphic summary of the results indicating luminescence (in relative light units) as a function of time (in days). The results indicate that pH of 7.2 to 7.5 slowed the reduction in luminescence better than other tested pH. A pH of 7.2 was most effective till day 28. After day 28, a pH of 7.5 was more effective.

Surprisingly, a pH of 6.0 provided the highest luminescence until day 14 followed by a sharp drop-off in luminescence.

### Example 8

### Yeast Extract Titration Results

In order to determine the effect of complex protein mixtures on luminescence during long term storage at refrigeration temperatures, storage solutions with yeast extract at concentrations of 0.01, 0.05 and 0.2% were tested. A medium with no added yeast extract served as a negative control. Assay of the effect of pH was as described in Example 1.

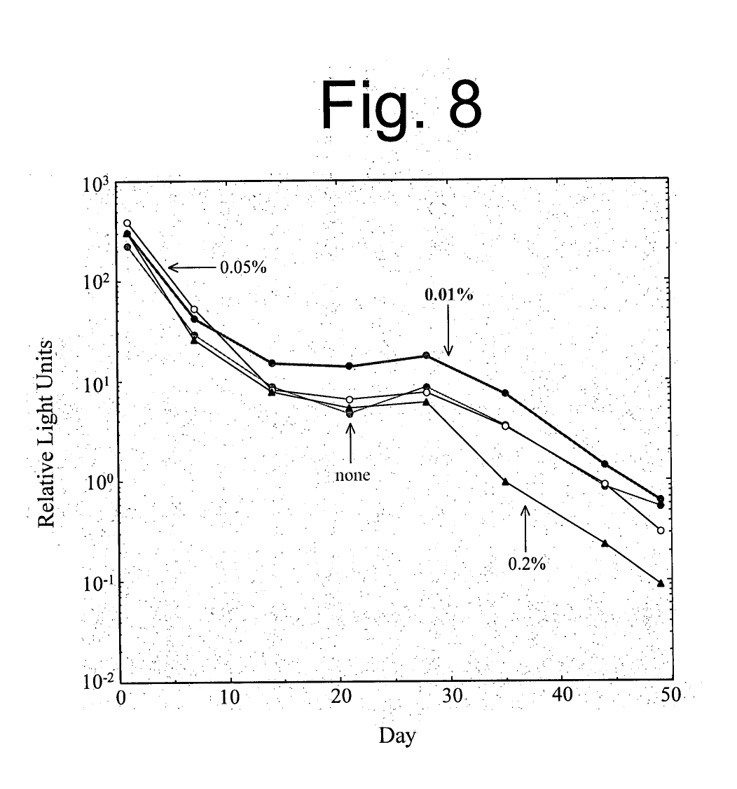

FIG. 8 provides a graphic summary of the results indicating luminescence (in relative light units) as a function of time (in days). The results indicate that 0.01% yeast extract slowed the reduction in luminescence better than other tested concentrations. When all other components were optimized, 0.05% yeast extract was selected.

### Example 9

### Effect of Different Antibiotics

In order to determine the effect of different antibiotics on luminescence during long term storage at refrigeration temperatures, storage solutions with ampicillin, chloramphenicol, neomycin and kanamycin at a concentration of 15 mg/L were tested. A medium with no added antibiotic served as a negative control. An additional medium containing all 4 antibiotics was also assayed (15 mg/L of each). Assay of the effect of antibiotics was as described in Example 1.

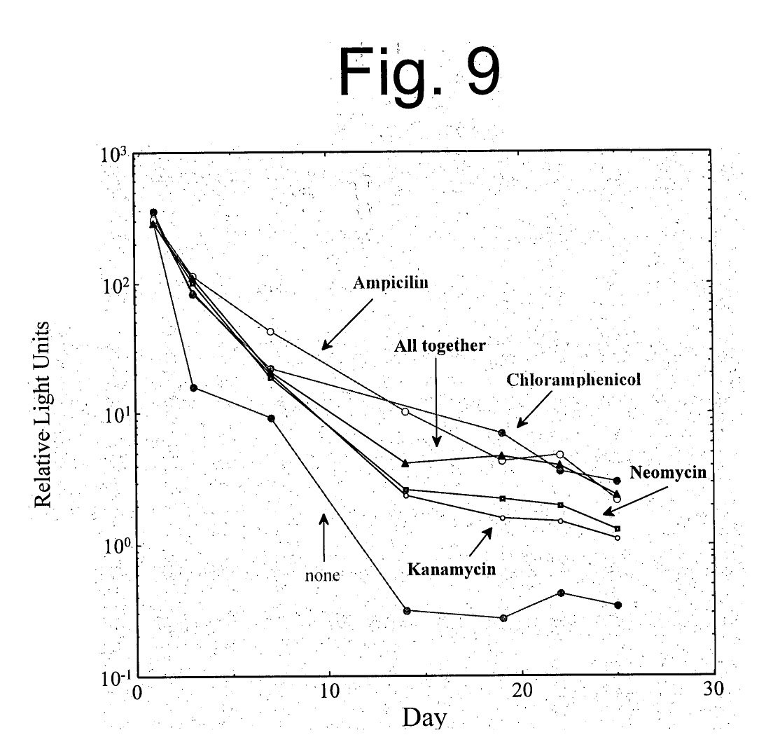

FIG. 9 provides a graphic summary of the results indicating luminescence (in relative light units) as a function of time (in days). The results indicate that ampicillin and chloramphenicol slowed the reduction in luminescence better than other tested antibiotics.

### Example 10

### Exemplary Medium and Ranges of Exemplary Components

Table 2 presents an exemplary medium recipe and exemplary ranges based upon results presented in examples 2 through 9.

TABLE 2
exemplary recipe and exemplary ranges of exemplary
components for bacterial medium according to various
embodiments of the invention

| component | solution | rangeI | range II |
|---|---|---|---|
| NaCl (%) | 2.0 | 2.0-3.0 | 1.0-3.0 |
| Inositol (%) | 5.0 | 5.0-7.0 | 0.5-7.0 |
| Mg (mM) | 2.0 | 1.0-200 | 0.0-300 |
| Yeast Extract (%) | 0.05 | 0.01-0.05 | 0.01-0.05 |
| BSA (%) | 0.05 | 0.62-1.0 | 0.02-1.0 |
| Ethanol (%) | 1.5 | 1.0-3.5 | 0.1-3.5 |

The column labeled “solution” indicates exemplary concentrations for a medium when all titration results were considered together.

The column labeled “range I” indicates concentrations that provided a marked improvement over negative control for the indicated component.

The column labeled “range II” indicates concentrations that produced no negative effect relative to negative control for the indicated component.

In some cases, it is preferable to reduce the concentration of Magnesium to 0.0 mM in the solution.

### Example 11

### General Titration Protocol

Assays for the presence of toxic substances based on bacterial luminescence measure a change from baseline after the sample to be tested is brought into contact with the bacteria. For this reason, it is possible to work with a suspension that does not contain a fixed density of viable cells over time. However, the initial baseline must be sufficiently high that a change can be measured.

In order to determine the cell density throughout the experimental period, serial dilutions of bacterial suspension from the exemplary medium shown in Table 2 were plated out and colonies were counted to determine viable cell density in the bacterial suspension. Results are presented in Table 3, below.

TABLE 3
viable cells as a function of time

| | Day 1 | Day 7 | Day 14 | Day 21 | Day 28 | Day 35 |
|---|---|---|---|---|---|---|
| Cells/ | 3 × 10⁸ | 1.5 × 10⁸ | 1.85 × 10⁸ | 7.7 × 10⁷ | 2.5 × 10⁷ | 1 × 10⁶ |
| mL | | | | | | |

Results presented in Table 3 indicate that the concentration of viable cells (P. leiognathi; E. coli) decreased by about 50% during the first 7 days, remained relatively stable up to day 21 and underwent a total reduction of one order of magnitude by 28 days and 2 orders of magnitude by 35 days. Table 3 indicates average numbers obtained from three repetitions for each bacterial strain.

Results summarized in Table 3 indicate that sufficient luminescence for assays of toxicity should be available for at least 28 to 35 days.

### Example 12

### Sensitivity as a Function of Time

In order to determine how long a bacterial suspension stored in a solution according to an exemplary embodiment of the invention could be useful in a toxicity assay, a toxicity assay (ToxScreen-II, CheckLight Ltd, Qiryat Tivon, Israel) was conducted repeatedly over time.

In the assay, 0.01 mL aliquots were drawn from the bacterial suspension (P. leiognathi) in the medium at 4 time points during 30 days and incubated with chemical toxicants in assay buffer diluted in mineral water. Light level was determined after 30 minutes at 27° C.

TABLE 4
Sensitivity (mg/L) of P. leiognathi to various toxicants

| | Day 1 | Day 7 | Day 18 | Day 30 |
|---|---|---|---|---|
| PbCl₂ | 2 | 2.5 | 3 | 2.5 |
| CdCl₂ | 0.2 | 0.25 | 0.3 | 0.4 |
| DDT | 0.007 | 0.006 | 0.004 | 0.006 |
| K₂Cr₂O₇ | 0.5 | 0.7 | 0.5 | 0.6 |

Results summarized in Table 4 indicate that the decrease in number of viable cells described in Example 11 does not interfere with the utility of the stored bacterial suspension as a substrate for a toxicity assay. In the experiments summarized in Table 4, results for each assayed toxicant were steady over time with a deviation expected from such bioassay.

### Example 13

### Use in Genetically Modified Bacteria

In order to demonstrate the utility of storage solutions according to exemplary embodiments of the invention in bacteria which are luminescent as a result of genetic manipulation, a titration of NaCl concentrations as described in Example 6 was conducted on E. coli harboring the luminescence system of V. fischeri (data not shown). A salt concentration of 0.8-0.85% was determined to be useful in these bacteria.

A variety of numerical indicators have been utilized to describe various components of the apparatus and/or relationships between the apparatus and a water supply. It should be understood that these numerical indicators could vary even further based upon a variety of engineering principles, materials, intended use and designs incorporated into the invention. Additionally, components and/or actions ascribed to exemplary embodiments of the invention and depicted as a single unit may be divided into subunits. Conversely, components and/or actions ascribed to exemplary embodiments of the invention and depicted as sub-units may be combined into a single unit with the described/depicted function.

Alternatively, or additionally, features used to describe a method can be used to characterize an apparatus and features used to describe an apparatus can be used to characterize a method.

It should be further understood that the individual features described hereinabove can be combined in all possible combinations and sub-combinations to produce exemplary embodiments of the invention. The examples given above are exemplary in nature and are not intended to limit the scope of the invention which is defined solely by the following claims. Specifically, the invention has been described in the context of a water supply but might also be used in analysis of other fluids.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A medium composition, the composition comprising about:

| | |
|---|---|
| NaCl/KCl (%) | 1.0-3.0; |
| Inositol/lactose/trehalose/dextran (%) | 0.5-7.0; |
| Mg/Ca (mM) | 0.0-300; |
| Yeast Extract/Casamino acids (%) | 0.01-0.05; |
| BSA/egg albumin (%) | 0.02-1.0; and |
| Ethanol/methanol/propanol (%) | 0.1-3.5. |

2. The medium composition of claim 1, comprising about:

| | |
|---|---|
| NaCl/KCl (%) | 2.0-3.0; |
| Inositol/lactose/trehalose/dextran (%) | 5.0-7.0; |
| Mg/Ca (mM) | 1.0-200; |
| Yeast Extract/Casamino acids (%) | 0.01-0.05; |
| BSA/egg albumin (%) | 0.02-0.1; and |
| Ethanol/methanol/propanol (%) | 1.0-3.5. |

3. The medium composition of claim 2, comprising about:

| | |
|---|---|
| NaCl/KCl (%) | 2.0; |
| Inositol/lactose/trehalose/dextran (%) | 5.0; |
| Mg/Ca (mM) | 2.0; |
| Yeast Extract/Casamino acids (%) | 0.05; |
| BSA/egg albumin (%) | 0.05; and |
| Ethanol/methanol/propanol (%) | 1.5. |

4. The medium composition of claim 1, comprising an antibiotic.

5. The medium composition of claim 1, provided as a liquid.

6. A bacterial suspension comprising the medium composition of claim 1 and bacteria suspended therein.

7. The bacterial suspension of claim 6, wherein said bacteria comprise luminescent bacteria.

8. A method of preparing a bacterial suspension according to claim 6, the method comprising:
wetting fibers with a bacterial culture to form wetted fibers;
lyophilizing the wetted fibers to form a plug; and
immersing the plug in the medium composition to form the bacterial suspension.

9. A method of storing bacteria, the method comprising:
(a) suspending bacteria in the medium composition of claim 1 so as to form a bacterial suspension; and
(b) storing said bacterial suspension under conditions for maintaining bacteria viability and preventing bacterial expansion.

10. The method of claim 9, wherein said suspending comprises immersing lyophilized bacteria on fibers in the medium composition.

11. The method of claim 9, wherein said bacteria comprise luminescent bacteria.

12. The method of claim 9, wherein said maintaining bacteria viability and preventing bacterial expansion is for at least 2 weeks at 2 to 8 degrees centigrade.

13. The method of claim 9, wherein the medium composition comprises at least one antibiotic.

14. The method of claim 13, wherein the at least one antibiotic comprises at least one compound selected from the group consisting of chloramphenicol and ampicillin.

15. The method of claim 13, wherein the at least one antibiotic is provided at a concentration of about 15 mg/L.

16. The method of claim 11, wherein said conditions further comprise maintaining luminescence of said luminescent bacteria.

17. An article of manufacture, comprising:
(a) a medium composition according to claim 1; and
(b) instructions for suspending bacteria in the medium composition to form a bacterial suspension and storing the bacteria at −2 to +8 degrees centigrade.
